(19) United States
(12) Patent Application Publication
(10) Pub. No.: US 2024/0189809 A1
(43) Pub. Date: Jun. 13, 2024

(54) SAFETY CABINET

(71) Applicant: Hitachi Industrial Equipment Systems Co., Ltd., Tokyo (JP)

(72) Inventors: Takeshi MATSUMURA, Tokyo (JP); Takeshi KANEKO, Tokyo (JP)

(73) Assignee: Hitachi Industrial Equipment Systems Co., Ltd., Tokyo (JP)

(21) Appl. No.: 17/615,969

(22) PCT Filed: Jun. 15, 2021
(86) PCT No.: PCT/JP2021/022743

§ 371 Date: Feb. 15, 2024

(51) Int. Cl. B01L 1/02; B01L 1/00

(52) U.S. Cl. CPC B01L 1/02; B01L 1/50; B01L 2200/082; B01L 2200/085

(57) ABSTRACT

A safety cabinet has an opening portion in the front surface of a work room and a front shutter and supplies clean air to the work room from above. A side slit is provided in the side surface of the work room, air from the provided side slit joins an air flow path suctioned from a front grille of a workbench forming the bottom surface of the work room, and the turbulence of an airstream can be prevented even when a device is installed in the work room.

### TECHNICAL FIELD

The present invention relates to a safety cabinet to be used for medicine development, research on pathogens, etc.

### BACKGROUND ART

When pathogens, etc. are handled for manipulation and observation of cells, research on pathogens such as viruses, medicine development such as vaccine development, etc., a safety cabinet is to be used.

As an example of a safety cabinet, Patent Document 1 discloses a safety cabinet having a work space where a worker works, a front shutter formed on the front surface of the work space, a work opening portion connected to the work space below the front shutter, and exhaust means for suctioning air from the work opening portion and exhausting the air in the work space to the outside of the safety cabinet via air cleaning means by a blower. A front shutter straightening plate inclined toward the inside of the work space is formed on the lower side of the front shutter, right and left side wall surfaces in the work space are provided, a side exhaust flow path is formed by the side wall surface and the side surface of the safety cabinet, slits are formed in the right and left side wall surfaces, and the air in the work space is sent to the side exhaust flow path via the slit and exhausted via the air cleaning means.

### CITATION LIST

### Patent Document

Patent Document 1: JP 2014-073457 A

### SUMMARY OF THE INVENTION

### Problems to be Solved by the Invention

By using the safety cabinet, it is possible to prevent contamination attributable to cell manipulation and observation and intra-work room pathogen handling and the like and prevent a pathogen or the like from leaking from the inside of the work space to the worker side.

Although the worker sits in front of the safety cabinet and performs the work with his or her arm inserted into the work space from the work opening portion below the front shutter as for the safety cabinet disclosed in Patent Document 1, Patent Document 1 does not consider performing work with a cell production machine such as a 3D bioprinter or a device such as a conveyance machine installed in the safety cabinet. In other words, the downflow wind speed of the entire work room in a case where a device is installed in the work room is not considered. In addition, the flow of air in the work space at a time when a device such as an incubator is connected to only one of the right and left sides of the main body of the safety cabinet is not considered.

An object of the present invention is to provide a safety cabinet preventing contamination of a cell, a pathogen, or the like when a cell production machine such as a 3D bioprinter or a device such as a conveyance machine is installed in the safety cabinet.

In addition, an object of the present invention is to provide a safety cabinet preventing contamination of a cell, a pathogen, or the like when a device such as an incubator is connected to only one of the right and left sides of the main body of the safety cabinet.

Further, an object of the present invention is to provide a safety cabinet capable of preventing a cell, a pathogen, or the like from leaking toward a worker from the inside of a work room.

### Solutions to Problems

An example of “safety cabinet” of the present invention for solving the above problems includes: a work room formed inside a front shutter, the safety cabinet suctioning air in the work room, cleaning the air, supplying the air to the work room in part, and exhausting the air in part; a workbench configuring a bottom surface of the work room; a lower flow path configured by the workbench and a part of the safety cabinet; a side flow path configured by a side surface of the work room and a part of a side surface of the safety cabinet; and a back flow path configured by a back surface of the work room and a part of a back surface of the safety cabinet. A circulation flow path is formed by at least the lower flow path, the side flow path, and the back flow path. The safety cabinet further includes a slit in the side surface of the work room. An airstream from the work room is suctioned from side slits, passes through a side chamber, and joins the lower flow path.

### Effects of the Invention

According to the present invention, the side slit is provided in the side surface of the work room and an exhaust airstream from the work room joins an air flow path suctioned from a front grille of the workbench. As a result, clean air can be exhausted from the work room without providing a pressure difference around a connecting portion and contamination in the work room can be suppressed even when a device is connected to either the right or left side of the main body of the safety cabinet.

In addition, the cleanliness of the work room can be maintained by exhausting dust generated by a worker and dust generated as a result of device operation from the side slit to an exhaust circulation flow path.

Tasks, configurations, and effects other than those described above are apparent from the description of the following embodiment.

### BRIEF DESCRIPTION OF THE DRAWINGS

FIG. 1A is a front view of a safety cabinet.

FIG. 1B is a cross-sectional side view of the safety cabinet.

FIG. 2 is a cross-sectional perspective view of the safety cabinet.

FIG. 3A is a front view of the safety cabinet.

FIG. 3B is a cross-sectional side view of the safety cabinet.

FIG. 4 is a diagram illustrating details of a work room of the safety cabinet.

### MODE FOR CARRYING OUT THE INVENTION

### First Embodiment

Hereinafter, an embodiment of the present invention will be described with reference to the drawings. It should be noted that the same constituent element in the drawings for describing the embodiment is given the same name and reference numeral without redundant description.

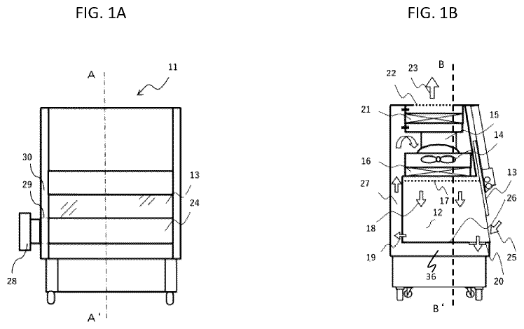

The operation of a safety cabinet will be briefly described before the detailed description of the embodiment. FIG. 1A illustrates a schematic front view of the safety cabinet, and FIG. 1B illustrates a schematic side view of the safety cabinet in which the A-A′ cross section of FIG. 1A is viewed from the left.

Although the safety cabinet includes various cabinets for regenerative medical treatments, industrial purposes, etc., a safety cabinet to be used for medicine development or research pathogens, etc. will be specifically described in the on following description.

A work room 12, where a worker performs work with his or her hand in, is provided in the safety cabinet housing that configures a safety cabinet 11. A front shutter 13 sliding up and down to open and close is provided on the front surface of the work room 12. The front shutter 13 is made of a transparent material such as glass and hard plastic, and the inner portion of the front shutter 13 can be seen. A pressure chamber 15 is pressurized when a safety cabinet fan 14 is operated. A blowout HEPA filter 16 is connected to the pressure chamber 15. Dust in the pressure chamber 15 is filtered by the blowout HEPA filter 16, and clean air is blown out, straightened by a blowout straightening plate 17, and then supplied into the work room 12 as a blowout airstream 18. A plurality of rear slits 19 suctioning the air in the work room are provided on the back surface side of the work room 12. A plurality of front slits 20 are provided in the front surface of the bottom surface (workbench surface) of the work room 12 below the front shutter 13.

An exhaust HEPA filter 21 is also connected to the pressure chamber 15. The air pressurized in the pressure chamber 15 is filtered by the exhaust HEPA filter 21, passes through a safety cabinet exhaust port 22, and is exhausted from the safety cabinet 11 as exhaust air 23. Air equal in amount to the air exhausted from the safety cabinet 11 enters the safety cabinet 11. The air is an inlet airstream 25 generated in a work opening portion 24 below the front shutter 13.

The inlet airstream 25 is suctioned into the front slit 20 together with a part of the blowout airstream 18 in the work room 12. This air passes through a lower flow path 36 configured by a workbench 26 and a part of the main body of the safety cabinet. The blowout airstream 18 supplied to the work room 12 is suctioned by the front slit 20 and joins the lower flow path 36 described above. A part of the blowout airstream 18 is suctioned from the rear slit 19 formed in the surface of the work room 12 that is opposite to the front shutter 13 (facing surface), passes through a back flow path 27, and is suctioned into the safety cabinet fan 14.

The lower flow path 36 is formed by the lower side of the workbench 26 and a part of the main body of the safety cabinet 11. The back flow path 27 is formed by the back surface side of the work room 12 and a part of the outer portion (back surface) of the safety cabinet. In addition, a side flow path 30 is formed by the side surface of the work room 12 and a part of the side surface of the main body of the safety cabinet 11.

A part of the airstream passing through the circulation flow path including the lower flow path 36, the back flow path 27, and the side flow path 30 is suctioned into the safety cabinet fan 14 and discharged from the safety cabinet 11 as the exhaust air 23.

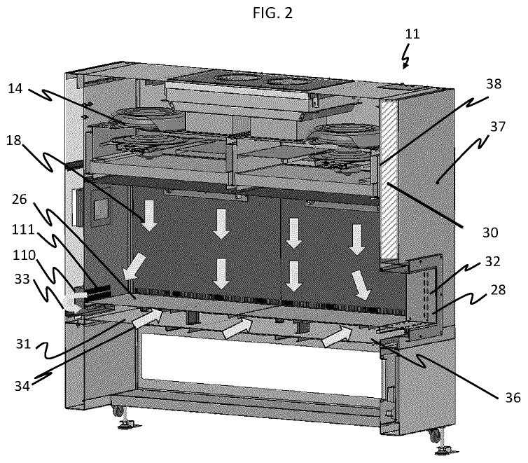

A connecting portion 28 connects the safety cabinet body 11 and a device outside the safety cabinet such as an incubator. The connecting portion 28 forms an air barrier so that contaminated air does not enter the work room 12 from the outside of the safety cabinet 11. A connecting portion slit 32 (see FIG. 2) is provided around the connecting portion 28 so that the air barrier is formed. The connecting portion slit 32 is provided with the side flow path 30 in the entire side surface of the safety cabinet 11 so as to surround the connecting portion 28. The contaminated air from the outside of the safety cabinet 11 and a part of the blowout airstream 18 in the work room 12 pass through the side flow path 30 and are suctioned into the safety cabinet fan 14. If the connecting portion 28 connecting an incubator or the like is provided on either the right or left side of the safety cabinet, the blowout airstream 18 in the work room 12 may be biased to the connecting portion side.

In addition, when a device such as a cell production machine and a conveyance machine is installed in the work room 12, dust may be generated from a movable portion of the device and the blowout airstream 18 is disturbed by the device installed in the work room 12.

In the safety cabinet of the related art, the blowout airstream 18 in the work room 12 may be biased to the connecting portion side if the connecting portion 28 connecting an incubator or the like is provided on either the right or left side of the safety cabinet.

In addition, the airstream is biased when a device such as a cell production machine and a conveyance machine is installed in the work room 12.

The airstream is further biased if a device such as a cell production machine and a conveyance machine is installed in the work room 12 with the connecting portion installed on either the right or left side of the safety cabinet. Dust or the like staying in the work room 12 results in an increase in the chance of touching a worker. Accordingly, contamination may arise and the performance of the safety cabinet may not be retained. Further, the bias of the blowout airstream 18 may be further promoted by an increase in the distance from a dust source such as the machine installed in the work room 12 to the front slit 20 or the rear slit 19 and the performance of the safety cabinet may not be retained.

In the present embodiment, the safety cabinet is provided with a circulation flow path including a side slit 111 in addition to the front slit 20, the rear slit 19, and the connecting portion slit 32 and is capable of suctioning the blowout airstream 18 from the work room 12 and collecting dust from a movable portion of a device.

In other words, by providing the side slit 111 in the surface of the work room 12 that opposes the connecting portion 28, an airstream is suctioned from the work room 12 by the side slit 111 and joins the lower flow path 36 suctioned from the front slit 20 of the workbench 26. As a result, clean air can be exhausted from the work room and contamination in the work room can be suppressed.

FIG. 2 illustrates a schematic perspective view of the safety cabinet of the present embodiment in which the B-B′ cross section of FIG. 1B is diagonally viewed from the right side.

The connecting portion 28 is necessary in connecting the safety cabinet 11 and an incubator or the like. Although FIG. 2 illustrates an example in which the connecting portion 28 is provided on the right side of the safety cabinet 11, the connecting portion may be provided on the left side.

The connecting portion 28 is provided with a connecting portion intake slit 32 around the connecting portion 28 so that an air barrier is formed and contaminated air does not enter the work room 12. A part of the blowout airstream 18 suctioned from the connecting portion intake slit 32 passes through the side flow path 30 and is suctioned into the safety cabinet fan 14. The side flow path 30 is configured by a side surface 38 of the work room 12 and a part of a main body side surface 37 of the safety cabinet 11. A sealing material or the like for negative pressure maintenance is installed at the part of the side flow path 30 that is in contact with the connecting portion 28 such that no airstream leakage occurs.

The lower flow path 36 is configured by the lower surface of the workbench 26 and a part 31 of the safety cabinet main body and allows a lower surface airstream 34 to flow.

A side chamber 110 is provided on the left side of the safety cabinet 11, that is, the surface of the safety cabinet 11 facing the connecting portion 28, and a side airstream 33 suctioned from the side slit 111 is allowed to join the lower flow path 36. The side chamber 110 and the lower flow path 36 have the same pressure. As in the case of the connecting portion 28, a sealing material is installed in the side chamber 110 such that no airstream leakage occurs. The side chamber 110 can be smaller in size than the connecting portion 28, and thus the sealing material required for the side chamber 110 side can be smaller in amount than the sealing material required for the connecting portion 28 and the risk of airstream leakage can be reduced.

The side slit 111 is provided at a position lower than the height of, for example, a machine installed in the work room 12. Preferably, the side slit 111 is provided in the vicinity of the workbench 26. A necessary number of the side slits 111 are provided such that an area is attained that is required for suctioning the same amount of air as the air suctioned by the connecting portion intake slit 32 of the connecting portion 28 installed on the facing surface of the work room. In the example of FIG. 2, balance with the air suctioned from the connecting portion intake slit 32 is sought by the side slit 111 being configured to have one upper stage and one lower stage.

By providing the side slit 111 in this manner, an airstream is suctioned from the work room 12 by the side slit 111 and joins the lower flow path 36 suctioned from the front slit 20 of the workbench 26. As a result, contamination in the work room can be suppressed by the airstream biased to the connecting portion 28 flowing into the work room 12 in a well-balanced manner and clean air being exhausted from the work room.

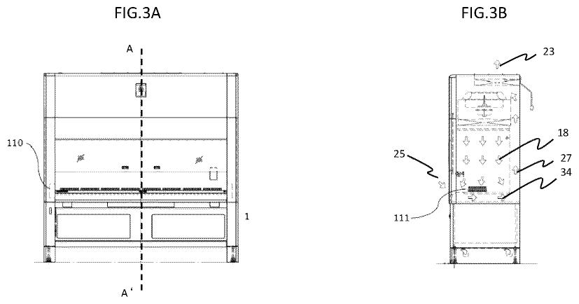

FIGS. 3A and 3B illustrate an example of the safety cabinet of the embodiment. FIG. 3A illustrates a cross-sectional view in which the safety cabinet is viewed from the front, and FIG. 3B illustrates a cross-sectional view of the safety cabinet in which the A-A′ cross section of FIG. 3A is viewed from the right.

FIG. 3A illustrates the configuration of the side chamber 110, and FIG. 3B illustrates the configuration of the side slit 111 and airstreams in the work room 12 of the safety cabinet 11 and a circulation flow path such as the lower flow path 36, the side flow path 30, and the back flow path 27.

A plurality of the side slits 111 are provided in the side surface of the work room 12. The blowout airstream 18 supplied to the work room 12 is suctioned by the side slit 111, joins the lower surface airstream 34 of the lower flow path 36 below the workbench 26 via the side chamber 110, and is suctioned into the safety cabinet fan 14 via the side flow path 30. Even in the rear slit 19 formed in the surface opposite to the front shutter 13, a part of the blowout airstream 18 is suctioned, joins the back flow path 27, and is suctioned into the safety cabinet fan 14.

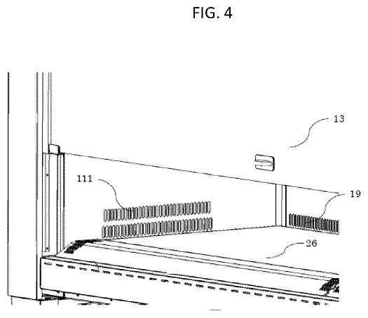

FIG. 4 is a perspective view illustrating details of the work room. The blowout airstream 18 suctioned into the side slit 111 in the side surface of the work room 12 passes through the side chamber 110 and joins the lower flow path 36 passing below the workbench 26.

The pressure is the same in the lower flow path 36 below the workbench 26 and in the side chamber 110. By providing the flow path of the same pressure, a side flow path does not have to be provided in the entire side surface of the safety cabinet unlike on the connecting portion side. In connecting another device on one of the right and left sides of the safety cabinet 11, turbulence of the blowout airstream 18 in the work room 12 can be suppressed by providing the side slit 111 such that the suction wind speed is close to that around the connecting portion.

When a device such as a cell production machine and a conveyance machine is installed in the work room 12, dust may be generated from a movable portion of the device.

In the safety cabinet of the related art, the front slit 20 and the rear slit 19 are provided and the blowout airstream 18 is suctioned from the front and back of the work room 12, and yet the blowout airstream 18 is biased on the right and left sides of the work room 12 as the blowout airstream 18 is also suctioned into the connecting portion 28 by the connecting portion slit 32. In the present embodiment, the side slit 111 is provided in the surface of the work room 12 that faces the connecting portion slit 32, and thus suction is also performed from the surface of the work room 12 that faces the connecting portion 28 and the bias of the blowout airstream can be prevented in the work room 12. In addition, the side slit 111 is capable of collecting dust from a part near a device installed in the work room 12.

According to the present embodiment, turbulence of the airstream in the work room can be prevented and contamination can be suppressed by the suction airstream from the side surface joining the flow path below the workbench surface. In addition, the airstream suctioned from the side slit 111 is allowed to flow into the lower flow path 36 by the side chamber 110, and thus a flow path does not have to be provided in the entire side surface of the safety cabinet.

In addition, when a device is installed in the work room, dust can be collected from a part close to the source of the dust and contamination caused by operation of the conveyance unit of the device or manual work by a person can be suppressed.

### REFERENCE SIGNS LIST

11 Safety cabinet
12 Work room
13 Front shutter
14 Safety cabinet fan
15 Pressure chamber
16 Blowout HEPA filter
17 Blowout straightening plate
18 Blowout airstream
19 Rear slit
20 Front slit
21 Exhaust HEPA filter
22 Exhaust port
23 Exhaust air
24 Work opening portion
25 Inlet airstream
26 Workbench
27 Back flow path
28 Connecting portion
29 Slit
30 Side flow path
32 Connecting portion slit
33 Side airstream
34 Lower surface airstream
36 Lower flow path
37 Main body side surface
38 Side surface of work room
110 Side chamber
111 Side slit

1. A safety cabinet comprising:
a work room formed inside a front shutter, the safety cabinet suctioning air in the work room, cleaning the air, supplying the air to the work room in part, and exhausting the air in part;
a workbench configuring a bottom surface of the work room;
a lower flow path configured by the workbench and a part of the safety cabinet;
a side flow path configured by a side surface of the work room and a part of a side surface of the safety cabinet;
a back flow path configured by a back surface of the work room and a part of a back surface of the safety cabinet;
a circulation flow path formed by at least the lower flow path, the side flow path, and the back flow path; and
a slit in the side surface of the work room,
wherein an airstream from the work room is suctioned from side slits, passes through a side chamber, and joins the lower flow path.

2. The safety cabinet according to claim 1, comprising a side chamber allowing the airstream suctioned from the side slit to flow to the lower flow path,
wherein the side chamber and the lower flow path have the same pressure.

3. The safety cabinet according to claim 2, comprising a connecting portion for connection to an external device and a connecting portion slit for forming an air barrier in the connecting portion on a right or left side of the work room,
wherein the side slit and the side chamber are provided on a surface of the work room facing the connecting portion to seek right-left balance of the airstream in the work room.

4. The safety cabinet according to claim 2, wherein the side slit is provided at a position lower than height of a device installed in the work room.

5. The safety cabinet according to claim 3, wherein
the connecting portion slit is provided around the connecting portion and suctions a part of a blowout airstream in the work room, and
the number of the provided side slits is a number at which the same amount of blowout airstream as the connecting part slit is suctioned.

6. The safety cabinet according to claim 5,
the side slit is a configuration having one upper stage and one lower stage in the work room.
